(19) United States
(12) Patent Application Publication
(10) Pub. No.: US 2006/0130849 A1
(43) Pub. Date: Jun. 22, 2006

(54) Inflatable bumper for back surgery

(75) Inventor: Marvin S. Lieberman, New York, NY (US)

Correspondence Address: ABELMAN, FRAYNE & SCHWAB, 150 East 42nd Street, New York, NY, 10017-5612, US

(73) Assignee: ALVIMAR MANUFACTURING CO. INC.

(21) Appl. No.: 11/094,706

(22) Filed: Mar. 30, 2005

Related U.S. Application Data
(60) Provisional application No. 60/566,657, filed on Apr. 30, 2004.
(60) Provisional application No. 60/558,519, filed on Apr. 1, 2004.

(51) Int. Cl. A61G 15/00; A61F 5/37; A61F 13/00

(52) U.S. Cl. 128/845; 128/846

(57) ABSTRACT

A protective bumper device formed of: a pair of cylinders, each having outer wall surfaces which include on each of the cylinders a first sidewall surface extending in the lengthwise direction, the cylinders each having a central longitudinal axis, the cylinders being positionable with the first wall surfaces of the two cylinders respectively spaced apart and in facing relationship, a web of sheet material having top and bottom edges and opposite side edges, each of the side edges fixed to one of the first sidewall surfaces of the cylinders respectively.

### BACKGROUND OF THE INVENTION

1. Field of the Invention

This invention is in the field of medical devices for protection of a wound area on a person's back or chest due to surgery or injury, particular devices which establish barriers to prevent the wound area from being contacted or bumped.

2. Description of the Related Prior Art

Prior art devices to protect wound areas includes soft bandages, of course, plus added layers of cotton or gauge and adhesive tape. For more elaborate protection a hard shield or other panel or layer may be added outward of the bandage with appropriate tape means to secure the further device to the patent.

Unwanted contact with the wound or the wound area which may include a deep incision and stitches or staples, can result in further injury or certainly pain. Such unwanted contact may occur when the patient walks and is bumped by another person or thing, or when the patient sits and leans backward against a chair back or when the patient attempts to lie on his or her back.

Depending on the wound or injury, it may be impossible or undesirable to sleep on the person's side or stomach, leaving only positions where the body is erect against a chair back or horizontal against a bed.

Besides the danger of actual painful or dangerous contact with the wound area, the patients often experience high anxiety due to the fear that something like that will happen, leaving them afraid to move, or walk about or even lie down. This is particularly true of back surgery patients, but also true for many injured persons who have not had surgery.

There are great numbers of additional situations where there has been no specific back injury, wound or surgery, but the person's back hurts at a specific time or chronically, and relief is needed when the person sits or reclines. Similarly, there are persons who could benefit from a protective device against unknown and/or unpredictable situations that lead to back pain.

The present invention addresses the above-described concerns as described below.

### SUMMARY OF THE NEW INVENTION

For a person who has had back surgery the new invention protects the incision area of the person's back by establishing an air layer outward of the bandage covering the incision. This invention provides a simple, economic and effective solution. The new invention, furthermore is lightweight, easy to use, and is readily portable and usable both when secured to the patient's back and when secured to a chair where he or she might wish to lean.

A first embodiment of this invention is a device comprising a pair of inflatable tubes constructed of thin plastic sheet material joined by a web of sheet material extending between them. When this device is used the inflated tubes are generally parallel and spaced apart. These inflated tubes have diameter which is greater than the thickness or height of the bandage. The device is placed with one tube adjacent to each side of the wound, with the web of the device overlying the wound which is usually but not always covered by a bandage. The inflated tubes serve as protective bumpers and barriers, so that when a user leans against the back of a chair the wound is not contacted by the chair back. Instead pressure contact is made only with healthy, strong areas of the back laterally outward of the wound.

This device may be secured onto the chair back or onto the person's back. For securing to the person, the device may have a strap or simple adhesive strips that engage the person's back. This device is similarly applicable to a person's chest for wounds, surgery, and strains or for general protection. Also, this device can be used as a back rest or support to enhance comfort in car, plane, train and theater seats, even when there is no specific wound, surgery or other injury.

The inflatable tubes may have a variety of cross-sectional shapes which include but are not limited to circular, semicircular and oval. The preferred shape is semicircular because it includes a generally flat surface for stable engagement with the surface of the back, and a smooth round exposed surface which will not catch on things like edges of furniture or clothing, when this device is secured to a person's back and/or worn under a shirt.

For normal circumstances it is preferred to have the two tubular bumpers on opposite sides of the wound. The preferred structure is to have the two tubes joined by a web or bridge creating a single device where the distance between the tubes is fixed. With this arrangement the tubes cannot extend too far apart and fail to protect the wound. The web extending between the two tubes provides a further function, in that it creates an elevated roof above and spaced from the wound, and thus affords a barrier against contact from things the user might lean against or bump into or against things or people that may become dangerously close to the person's back and wound.

For ease and economy of structure the web is made of the same plastic sheet material as that used for the inflatable tubes and may even be part of a contiguous sheet formed into one unitary structure. Also, the web may be perforated with substantial apertures to allow better air circulation in the vicinity of the wound and to lighten the structure and to enhance its aesthetic appearance.

The web may be attached to the tubes at a variety of elevations, including at mid-height, or at the top or elsewhere to establish a desired distance from the wound. For ease and economy of construction, each tube has its own air valve as opposed to using a single valve on one tube with an air conduit between tubes.

In the preferred embodiment the bumpers are straight cylinders on opposite sides of the wound. If it becomes desired to provide bumpers also above and below the wound, the device can be modified to add inflatable tubes at the top and bottom, forming a generally rectangular shape, or to use one or more tubes defining a generally circular or oval donut shape. The web or roof would still extend between opposite sides as a protective barrier.

The plastic sheet may be selected from many commercial choices including PVC, and simply needs to be thin, light, flexible and airtight when sealed, and to be readily adaptable to standard heat sealing techniques, unless the manufacturer should choose to use adhesive bonding instead.

The protective bumper devices of various shapes for use with wounds or injuries in a person's back or chest or for use merely for comfort, have been described above as inflatable devices made of thin flexible sheet material. Such construction has the advantages of being inexpensive in manufacture, lightweight in use and when moved or stored, and comfortable in use. It has been determined that many of the benefits afforded by this invention with the inflatable construction are also available with similar devices where the inflatable tubes are replaced with soft or firm resilient cushion-like material or firm material in the form of cylinders, rods or beams which may be plastic or rubber or fiber or any material which will partially or fully surround the area to be protected and include the structural features required to provide the shape and functions described earlier. As with the inflatable tubes these solid cylinders, rods or beams may have a great variety of cross-sections, including round, half-round, oval, square, rectangular and others.

The description above broadly outlines the more important features of the invention in order that the following detailed description may be better understood. There are, of course additional features of the invention that will be described hereinafter and which will form the subject of the claims appended hereto. Those skilled in the art, however, will appreciate that the core concept which is described and amplified in this disclosure may readily be utilized or incorporated in structures other than those of the specific embodiments described herein.

### BRIEF DESCRIPTION OF THE DRAWINGS

FIG. 1 is a top front perspective view of a first embodiment of the new back wound protective bumper device;

FIG. 2 is a front elevational view thereof;

FIG. 3 is a sectional view taken along line 3-3 in FIG. 2;

FIG. 4 is a perspective view of a second embodiment of the device attached to a chair back;

FIG. 5 is a fragmentary front elevational view of a the new device in FIG. 4;

FIG. 6 is a sectional view taken along line 5-5 of FIG. 5;

FIG. 7 is a front elevational review of a third embodiment of the new bumper device.

FIG. 7A is a bottom plane view thereof;

FIG. 8 is a front elevational view of a forth embodiment of the new bumper device;

FIG. 8A is a bottom plan view thereof;

FIG. 9 is a front elevational view of a fifth embodiment of the new bumper device; and

FIG. 9A is a bottom plan view thereof.

### DETAILED DESCRIPTION OF THE PREFERRED EMBODIMENTS

In the descriptions below similar elements of the versions embodiments are given the same reference numbers for convenience and clarity. The first embodiment 10 of the new back and chest wound protective bumper device as seen in FIGS. 1-3 comprises a pair of inflatable bumpers 12, 14, each having outer surfaces 15, 15A and inner surfaces 16, 16A, a web 18 extending between said inner surfaces 16, 16A, and tabs 20, 20A extending from said outer surfaces. Each tube 12, 14 in cross-section is generally semicircular, and the web 18 and tabs 20, 20A are generally rectangular. This device is formed essentially of two thin plastic sheets 22, 24 joined along heat seal lines to define the two tubular elements 12, 14, the central web 18, and the outer tabs 20, 20A. More specifically, heat seal lines 25, 26, 27 and 28 establish the elongated sides of the tubes 12, 14, and heat seal lines 25, 29 and 27, 30 define the elongated sides of the tabs 20, 20A. Additional transverse heat seal lines 31, 32 at the top and bottom respectively further define the web and tabs. Finally, there are top end pieces 33, 33A and bottom end pieces 34, 34A which establish the semicircular shape of the cross-section of the tubes. The end pieces are made of the same plastic sheet and are heat sealed similarly as said previously described sheets. Each tube has its own inlet/outlet valve 36, 36A respectively for receiving air to inflate and releasing air to deflate. The central web 18 is optimally perforated by apertures 40 to render it lighter and to enhance the aesthetic appearance.

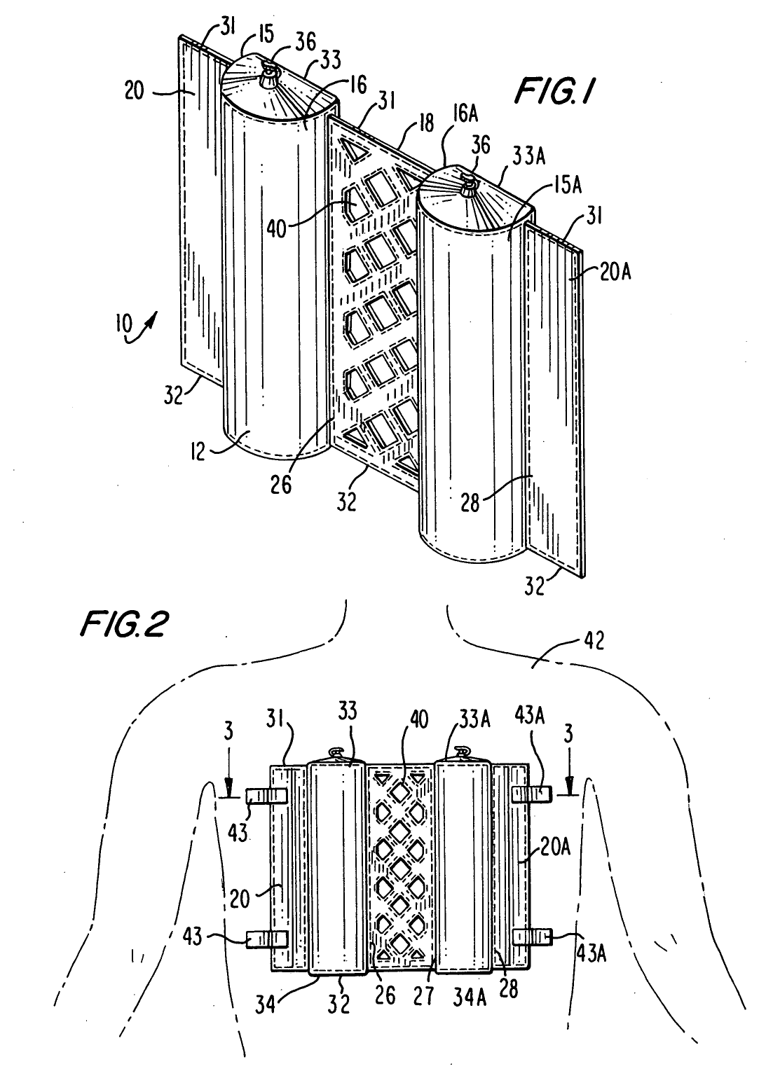

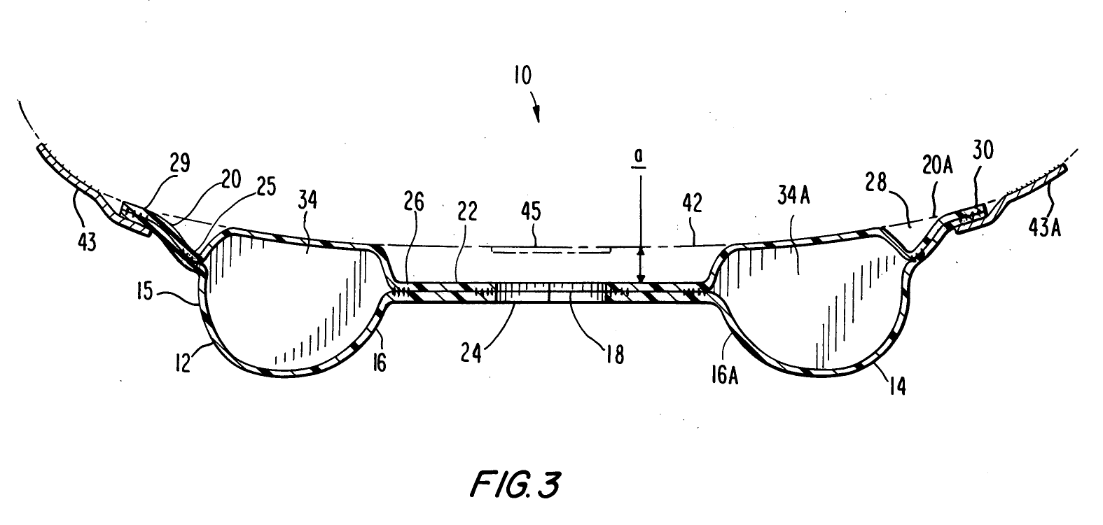

FIGS. 2 and 3 illustrate the device 10 in use where it is attached to a person's back 42 with small adhesive strips 43, 43A. FIG. 3 further indicates the wound area 45, which may or may not be covered by a bandage, depending on the degree of healing already achieved. As evident in FIG. 3 the web 18 is spaced outwardly from the wound area by a distance a, which is established by the location of the web 18 relative to the base line L-L of the semicircular form of the tubes. This web 18 thus forms a protective roof or wall spaced apart from the wound 45, or bandages thereon. The height of this roof, as indicated by distance a, can be varied by predetermined placement of the web relative to the sidewalls of the tubes.

The construction shown, comprising only two sheets 22, 24 and two end pieces 33, 33A, is extremely simple and economical to make; however, the device could optionally be made of more separate pieces of plastic sheet, and/or the heat seals could be varied in size and placement.

As seen, the inflatable tubes 12, 14 not only support the protective roof 18, but serve as protective bumpers to bar anything from approaching the wound from the sides, and the bumpers and the roof bar intrusion from the area in front of the device. When placing this device on one's back, as illustrated herein, a user can lean against a chair back or against a wall or lie on a bed without danger of the wound being contacted. This prevents the likely pain or further injury that would result from such contact, and furthermore eliminates the fear and anxiety of the patient about the possibility of the wound being contacted. As noted earlier, the device is useable on or for the chest also, and usable in connection with stationary furniture and/or with seats of cars, planes and trains, whether the user is injured or merely seeks the comfort and/or support provided by this device.

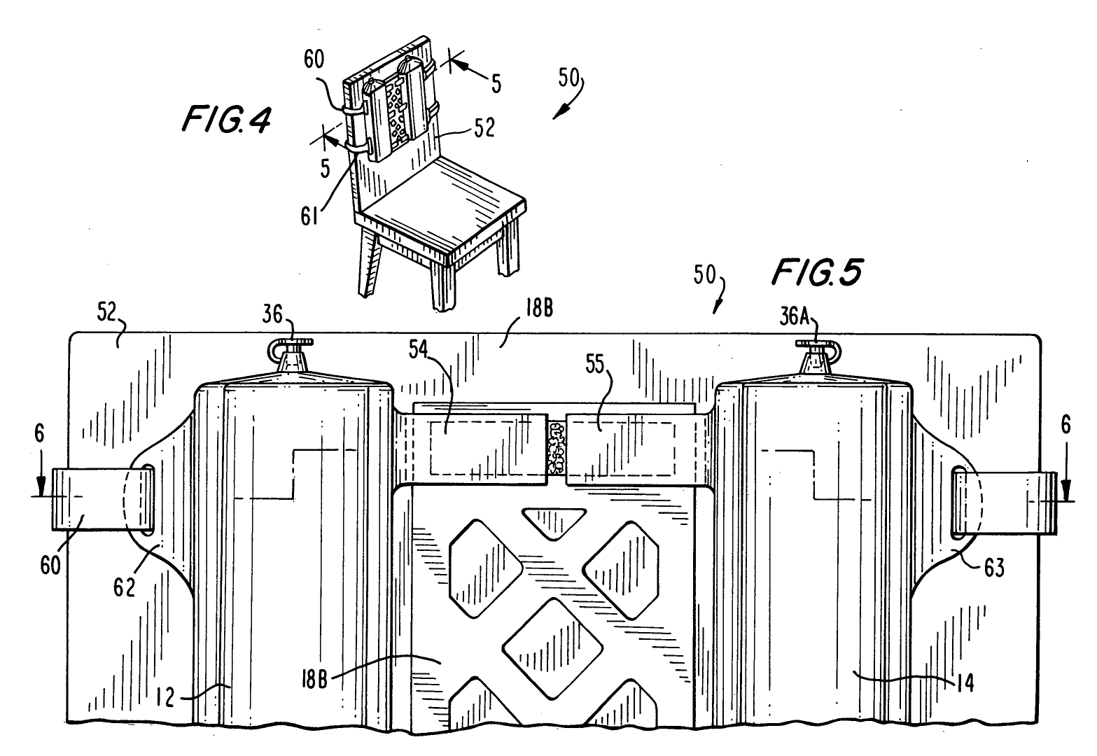

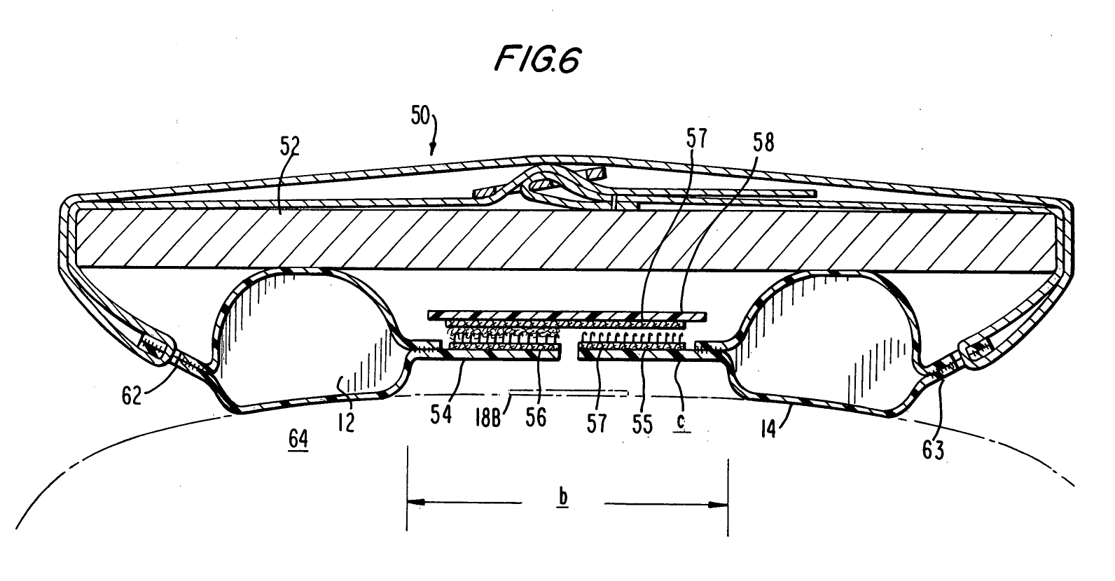

FIGS. 4-6 illustrate a second embodiment 50 of this device, and the use of this device secured to a chair's back 52 instead of being secured to a patient's back. The device as seen in FIGS. 4-6 differs from that of FIGS. 1-3 essentially in the construction of web 18B. FIG. 6 shows this web 18B formed of inner strips 54, 55 extending from tubes 12, 14 respectively, each of these strips including a layer of Velcro® 56, 57 on its front surface. These inner strips 54, 55 are totally separate and independent of each other but are coupled by overlying strip 58, which has a mating Velcro® strip 59, which is releasably coupleable to the Velcro® strips 56, 57. By this arrangement the tubes 12, 14 with said inner strips 54, 55 can be spaced apart a selected distance b, and maintained at this distance by the overlying strip 58 coupled to inner strips 54, 55. Obviously, by virtue of the characteristics of Velcro® tape, this coupling is releasable to allow repositioning of the two tubes at selected distances represented by b. Thus, with this embodiment, one device can be adjusted to fit persons of different sizes.

As seen in FIGS. 5 and 6, this device 50 is releasably secured to a chair back 52 by straps 60, 61 connected to tabs 62, 63, and the person's back 64 is spaced apart from the web 18B by distance c.

A further embodiment is possible by not fully inflating the tubes, which will result in tubes that are softer and more cushiony and extend a lesser distance c from the user's back. Also possible are tubes of a smaller diameter and tubes of other cross-sectional shapes, including but not limited to round, rectangular, oval, etc. For both embodiments, releasable coupling means other than Velcro® are possible. Also in both embodiments, the valves 36, 36A are standard and well known in the prior art.

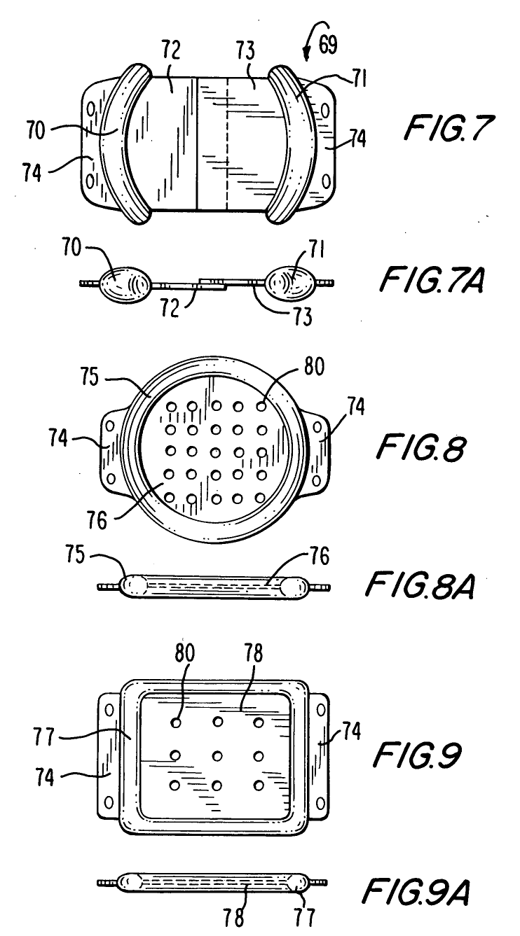

FIGS. 7, 7A, 8, 8A, 9 and 9A illustrate additional embodiments of this invention. In FIGS. 7 and 7A the device 69 comprises a pair of crescent shaped tubes 70, 71 joined by an adjustable web of parts 72, 73. Optional tabs 74 are for engaging and securing this device to a person or a chair.

In FIGS. 8 and 8A the device 75 is generally circular, and a web 76 is optional. This device has similar tabs 74.

In FIGS. 9 and 9A the device 77 is generally rectangular and the web 78 is optional. The device has similar tabs 74.

The device 69 is made similarly as the embodiment of FIGS. 4-6 except that the web 72, 73 comprises only two elements. The webs when included in the devices of FIGS. 8 and 9 are plain flat sheets perforated with ventilation holes 80.

The drawings herein attached and described disclose embodiments of the present invention which are constructed of sheet material in configurations that are inflatable and deflatable. As discussed earlier, the back bumper concept may be achieved also with solid or resilient cushion or with firm elements that substitute for the inflatable tubes seen in FIGS. 1-9A. In both the inflatable and the solid element embodiments the web may be made from a great variety of solid, apertured and/or size-adjustable elements.

While the present invention has been described with respect to what is presently considered to be the preferred embodiment, it is to be understood that the invention is not limited to the specifics of the embodiment described herein. The present invention is intended to cover various and equivalent arrangements included within the spirit and scope of the appended claims.

1. A protective bumper device comprising:
a. a pair of inflatable tubes made of flexible sheet material, each tube having outer wall surfaces which include on each of said tubes a first sidewall surface extending in the lengthwise direction,
said tubes each having a central longitudinal axis, said tubes being positionable with said first wall surfaces of said two tubes respectively spaced apart and in facing relationship,
b. a web of sheet material having top and bottom edges and opposite side edges, each of said side edges fixed to one of said first sidewall surfaces of said tubes respectively, and
c. valve means coupled to said tubes for inflation and deflation of said tubes.

2. A device according to claim 1 wherein said pair of tubes are positionable with their central longitudinal axes in generally parallel relationship.

3. A device according to claim 2 wherein each of said tubes is an elongated straight cylinder of generally uniform cross-section, and said web is generally rectangular.

4. A device according to claim 3 wherein said cross-section of each of said tubes is generally semicircular, thus defining a generally flat base and a curved top, aid tubes being positionable with their bases being generally coplanar, defining a first plane and with said web defining a second plane generally parallel to and spaced apart from said first plane.

5. A device according to claim 2 wherein said two tubes and said web are formed of two sheets of plastic in overlying relationship and joined along heat seal lines which define said two tubes and said web in-between said tubes.

6. A device according to claim 5 wherein each of said tubes further comprises one end cap segment of sheet material forming closure of each of the ends of each of said tubes, said end caps being heat sealed to said tubes respectively.

7. A device according to claim 2 wherein each of said tubes has a second wall surface generally opposite said first wall surface, said device further comprising an outer strip of sheet material extending from each of said second wall surfaces.

8. A device according to claim 7 wherein each of said outer strips extends lengthwise of one of said tubes.

9. A device according to claim 7 further comprising strap means extending from said outer strips.

10. A device according to claim 2 wherein said inflatable tubes together define a generally rectangular frame with spaced top and bottom tubular elements connected to said spaced apart side tubular elements.

11. A protective device for a person's lumbar back area, where the person's back defines a contact surface, a portion of which is wounded and is protected from pressure contact, comprising:
a. a pair of inflatable tubes having outside diameter D when inflated and length L,
b. a web of sheet material having length and having width terminating in opposite side edges,
c. each of said tubes secured along its length to one of said side edges of said web and extending generally parallel to the length of said web,
d. said web being extendible as a first plane between said tubes where said bottom surfaces of said tubes define a second plane spaced apart from said first plane, and
e. valve means for inflating and deflating each of said tubes.

12. A device according to claim 11 wherein said web joins said tubes at said second plane.

13. A device according to claim 11 wherein said web joins said tubes at an elevation intermediate said first and second planes.

14. A device according to claim 13 wherein said web joins said tubes at an elevation closer to said first plane than to said second plane.

15. A device according to claim 11 wherein each of said tubes has a generally flat top surface and a generally circular bottom surface.

16. A device according to claim 11 wherein each of said tubes has a generally circular cross section.

17. A device according to claim 11 where said tubes each comprise top and bottom sheets heat sealed together, and said web comprises top and bottom sheets heat sealed together, and said top sheets of said tubes and of said web comprises a contiguous sheet, and said bottom sheets of said tubes and of said web comprise a contiguous sheet, and said tubes are defined by heat seals formed between said top and bottom sheets.

18. A protection device for a person's lumbar back area, where the person's back defines a contact surface, a portion of which is wounded and is to be protected from contact, comprising:
a. a top sheet having length and width,
b. a bottom sheet having generally the same dimensions,
c. the opposite side edges being heat sealed together, a heat seal line parallel to and spaced from each of said side edges, thus defining a tube between said side edge and said heat seal line, said heat seal lines being spaced from each other, the ends of each tube being closed, and
d. valve means for inflating and deflating each of said tubes.

19. A bumper device comprising an inflatable tube defining a closed loop surrounding a space, said tube made of flexible sheet material, a web extending across at least a part of said space and secured to inner wall surfaces of said loop, and valve means coupled to said tube for inflation and deflation of said tube.

20. A bumper device comprising an elongated cylinder formed into a closed loop surrounding a space and a web extending across at least a part of said space and secured to inner wall surfaces of said loop.

21. A bumper device according to claim 20 wherein said elongated cylinder comprises a comprisable and flexible material.
